(19) United States
(12) Patent Application Publication
(10) Pub. No.: US 2011/0129944 A1
(43) Pub. Date: Jun. 2, 2011

(54) WATER-SOLUBLE NANOCRYSTALS AND METHODS OF PREPARING THEM

(75) Inventors: Fuke Wang, Singapore (SG); Mingyong Han, Singapore (SG)

(73) Assignee: Agency for Science, Technology and Research, Centros (SG)

(21) Appl. No.: 11/813,906

(22) PCT Filed: Jan. 17, 2005
(86) PCT No.: PCT/SG2005/000009

§ 371 Date: May 5, 2008

(51) Int. Cl. G01N 33/544; C07F 9/28; C08B 37/16

(52) U.S. Cl. 436/529; 556/21; 536/103

(57) ABSTRACT

Disclosed is a water soluble nanocrystal having a core comprising at least one metal M1 selected from an element of subgroup IIb, subgroup VIIa, subgroup VI11a, subgroup 1b, subgroup IV, main group II or main group III of the periodic system of the elements (PSE), at least one element A selected from an element of the main group V or VI of the periodic system of the elements, wherein a capping reagent is attached to the surface of the core of the nanocrystal, and wherein the capping reagent forms a host guest complex with a water soluble host molecule. Also disclosed is a water soluble nanocrystal having a core comprising at least one metal M1 selected from an element of subgroup I1b, subgroup VI1a, subgroup VI11a, subgroup 1b, subgroup IV, main group II or main group III of the periodic system of the elements (PSE), and at least one element A selected from an element of the main group V or VI of the periodic system of the elements, wherein a capping reagent is attached to the surface of the core of the nanocrystal, and wherein the capping reagent is covalently linked to a water soluble host molecule. Also disclosed is a water soluble nanocrystal having a core comprising at least one metal M1 selected from an element of subgroup I1b, subgroup VI1a, subgroup VI11a, subgroup 1b, subgroup IV, main group II or main group III of the periodic system of the elements (PSE), wherein a capping reagent is attached to the surface of the core of the nanocrystal, and wherein the capping reagent forms a host guest complex with a water soluble host molecule. Finally, compositions and uses of such nanocrystals are disclosed.

The invention relates to novel water-soluble nanocrystals and to methods of making the same. The invention also relates to uses of such nanocrystals, including but not limited to, in various analytical and biomedical applications such as the detection and/or visualization of biological materials or processes, e.g., in tissue or cell imaging, in vitro or in vivo. The present invention also relates to compositions and kits containing such nanocrystals which can be used in the detection of analytes such as nucleic acids, proteins or other biomolecules.

Semiconductor nanocrystals (quantum dots) have been receiving great fundamental and technical interest for their use in light-emitting devices (Colvin et al, Nature 370, 354-357, 1994; Tessler et al, Science 295, 1506-1508, 2002), lasers (Klimov et al, Science 290, 314-317, 2000), solar cells (Huynh et al, Science 295, 2425-2427, 2002) or as fluorescent biological labels in biochemical research areas such as cell biology. See for example, Bruchez et al, Science, Vol. 281, pages 2013-2015, 2001; Chan & Nie, Science, Vol. 281, pages 2016-2018, 2001; U.S. Pat. No. 6,207,392, summarized in Klarreich, Nature, Vol. 43, pages 450-452, 2001; see also Mitchell, Nature Biotechnology, pages 1013-1017, 2001, and U.S. Pat. Nos. 6,423,551, 6,306,610, and 6,326,144.

The development of sensitive nonisotopic detection systems for use in biological assays has significantly impacted many research and diagnostic areas, such as DNA sequencing, clinical diagnostic assays, and fundamental cellular and molecular biology protocols. Current nonisotopic detection methods are mainly based on organic reporter molecules that undergo color change or are fluorescent, luminescent. Fluorescent labeling of molecules is a standard technique in biology. The labels are often organic dyes that give rise to the usual problems of broad spectral features, short lifetime, photobleaching, and potential toxicity to cells. The recent emerging technology of quantum dots has spawned a new era for development of fluorescent labels using inorganic complexes or particles. These materials offer substantial advantages over organic dyes including large Stocks shift, longer emission half-life, narrow emission peak and minimal photo-bleaching (cf. references cited above).

Over the past decade, much progress has been made in the synthesis and characterization of a wide variety of semiconductor nanocrystals. Recent advances have led to large-scale preparation of relatively monodisperse quantum dots (Murray et al., J. Am. Chem. Soc., 115, 8706-15, 1993; Bowen Katari et al., J. Phys. Chem. 98, 4109-17, 1994; Hines, et al., J. Phys. Chem. 100, 468-71, 1996; Dabbousi, et al., J. Phys. Chem. 101, 9463-9475, 1997).

Further advances in luminescent quantum dot technology have resulted in an enhancement of the fluorescence efficiency and stability of the quantum dots. The remarkable luminescent properties of quantum dots arise from quantum size confinement, which occurs when metal and semiconductor core particles are smaller than their excitation Bohr radii, about 1 to 5 nm (Alivisatos, Science, 271, 933-37, 1996; Alivistos, J. Phys. Chem. 100, 13226-39, 1996; Brus, Appl Phys., A53, 465-74, 1991; Wilson et al., Science, 262, 1242-46, 1993). Recent work has shown that improved luminescence can be achieved by capping a size-tunable lower bandgap core particle with a higher band gap inorganic materials shell. For example, CdSe quantum dots passivated with a ZnS layer are strongly luminescence at room temperature, and their emission wavelength can be tuned from blue to red by changing the particle size. Moreover, the ZnS capping layer passivates surface nonradiative recombination sites and leads to greater stability of the quantum dot (Dabbousi et al., J. Phys. Chem. B101, 9463-75, 1997. Kortan, et al., J. Am. Chem. Soc. 112, 1327-1332, 1990).

Despite the progress in luminescent quantum dots technology, the conventional capped luminescent quantum dots are not suitable for biological applications because they are not water-soluble.

In order to overcome this problem, the organic passivating layer of the quantum dots was replaced with water-soluble moieties. However, the resultant derivatized quantum dots are less luminescent than the parent ones because of charge-carrier tunneling. (See, for example, Zhong et al., J. Am. Chem. Soc. 125, 8589, 2003). Short chain thiols such as 2-mercaptoethanol and 1-thio-glycerol have also been used as stabilizers in the preparation of water-soluble CdTe nanocrystals (Rogach et al., Ber. Bunsenges. Phys. Chem. 100, 1772, 1996; Rajh et al., J. Phys. Chem. 97, 11999, 1993). In another approach the use of deoxyribonucleic acid (DNA) as a water soluble capping compound is described (Coffer, et al., Nanotechnology 3, 69, 1992). In all of these systems, the coated nanocrystals were not stable and photoluminescent properties degraded with time.

In a further study, Spanhel et al. disclosed a Cd(OH)₂—capped CdS sol (Spanhel, et al., J. Am. Chem. Soc. 109, 5649, 1987). However, the colloids nanocrystals could be prepared only in a very narrow pH range (pH 8-10) and exhibited a narrow fluorescence band only at a pH of greater than 10. Such pH dependency greatly limits the usefulness of the material, in particular, such a nanocrystal is not suitable for use in biological systems.

In the International patent application WO 00/17656 core-shell nanocrystals are disclosed which are capped with a carboxyl acid or sulfonic acid compound of the formula SH(CH₂)ₙ—COOH and SH(CH₂)ₙ—SO₃H, respectively in order to render the nanocrystals water soluble. Similarly, the PCT application WO 00/29617 and British patent application GB 2342651 describe that organic acids such as mercapto acetic acid or mercaptoundecanoic acid are attached to the surface of nanocrystals to render them water soluble and suitable for conjugation of biomolecules such as proteins or nucleic acids. The GB patent application 2342651 also describes the use of trioctylphosphine as capping material that is to be supposed to confer water solubility of the nanocrystals.

The International patent application WO 00/27365 reports the use of diaminocarboxylic acids or amino acids as water-solubilising agents. The International patent application application WO 00/17655 discloses nanocrystals that are render water soluble by the use of a solubilising (capping) agent that has a hydrophilic moiety and a hydrophobic moiety. The capping agent attaches to the nanocrystal via the hydrophobic group whereas the hydrophilic group such as a carboxylic acid or methacrylic acid group provides for water solubility. In a further International patent application (WO 02/073155) water soluble semiconductor nanocrystals are described which use hydroxamates, derivatives of hydroxamic acid or multidentate complexing agents such as ethylenediamine as water-solubilising agents. Finally, the International patent application PCT WO 00/58731 discloses nanocrystals which are used for the analysis of blood cell populations and in which amino-derivatized polysaccharides having a molecular weight from about 3,000 to about 3,000,000 are linked to the nanocrystals.

However, despite these developments there remains a need for luminescent nanocrystals that can be used for detection purpose in biological assays. In this respect, it would be of helpful to have nanocrystals that can be attached to a biomolecule in such a manner that preserves the biological activity of the biomolecule. Furthermore, it would be desirable to have water-soluble semiconductor nanocrystals which can be prepared and stored as stable, robust suspensions or solutions in aqueous media. Finally, these water-soluble nanocrystals quantum dots should be capable of energy emission with high quantum efficiencies, and should possess a narrow particle size.

Accordingly, it is an object of the invention to provide nanocrystals that meet the above needs.

This object is solved by the nanocrystals and the processes of producing nanocrystals having the features of the respective independent claims.

In one embodiment, such a nanocrystal is a water soluble nanocrystal having a core comprising

at least one metal M1 selected from an element of subgroup Ib, subgroup IIb, subgroup IIIb, subgroup IVb, subgroup Vb, subgroup VIb, subgroup VIIb, subgroup VIIIb, main group II, main group III or main group IV of the periodic system of the elements (PSE),

wherein a capping reagent is attached to the surface of the core of the nanocrystal, and

wherein the capping reagent forms a host guest complex with a water soluble host molecule. Accordingly, in this embodiment the present invention is directed to a new class of water-soluble nanocrystal having a pure metal core.

In another embodiment, a nanocrystal of the invention is a water soluble nanocrystal having a core comprising at least one metal M1 selected from an element of subgroup Ib subgroup IIb, subgroup IVb, subgroup Vb, subgroup VIb, subgroup VIIb, subgroup VIIIb IIB-VIB, IIIB-VB or IVB, main group II, main group III or main group IV of the periodic system of the elements (PSE),
at least one element A selected from an element of the main group V or VI of the periodic system of the elements,
wherein a capping reagent is attached to the surface of the core of the nanocrystal, and
wherein the capping reagent forms a host-guest complex with a water soluble host molecule.

In another embodiment of the invention such a nanocrystal is a water soluble nanocrystal having a core comprising at least one metal M1 selected from an element of subgroup IIB-VIB, IIIB-VB or IVB main group II or main group III of the periodic system of the elements (PSE), and at least one element selected from an element of the main group V or VI of the periodic system of the elements, and,

wherein a capping reagent is attached to the surface of the core of the nanocrystal, and

wherein the capping reagent is covalently linked to a water soluble host molecule, and wherein the host molecule is selected from the group consisting of carbohydrates, cyclic polyamines, cyclic dipeptides, calixarenes, and dendrimers.

In yet another embodiment, the nanocrystal is a water soluble nanocrystal having a core comprising

at least one metal M1 selected from an element of subgroup IIb, IIB-VIB, IIIB-VB or IVB, main group II or main group III of the periodic system of the elements (PSE), and at least one element A selected from an element of the main group V or VI of the periodic system of the elements, and, wherein a hydrophobic capping reagent is attached to the surface of the core of the nanocrystal, and

wherein the hydrophobic capping agent is covalently linked to a crown ether and wherein the hydrophobic reagent has the formula (I)

HₐX—Y—Z,

wherein

X is a terminal group selected from S, N, P, or O═P,

A is an integer from 0 to 3,

Y is a moiety having at least three main chain atoms, and

Z is a hydrophobic ending group.

Accordingly, the invention is based on the finding that host molecules can be used to modify the surface properties of (semiconductor) nanocrystals such that the nanocrystals are readily soluble in water, and yet maintain a high physical and chemical stability in aqueous media. In addition, it has been found here that such host molecules, e.g. but not limited to, dendrimers, calixarenes or carbohydrates such as cyclodextrins, typically have a rather large hydrophobic internal cavity (although host molecules used in the invention can also have a rather hydrophilic cavity) that enables them to accept a wide range of organic molecules as guest. Accordingly, host molecules having a hydrophobic (or hydrophilic) cavity are suitable for forming host guest complexes with hydrophobic (or hydrophilic) reagents that are used for surface modification of quantum dots. Furthermore, such host molecules are also able to form host guest complexes with numerous compounds (linking agents) that are typically used for the conjugation of biological probes, thus offering a new and elegant route to biomolecular conjugates of luminescent nanocrystals that are suitable for numerous biological applications. In addition, host molecules may contain a number of solvent exposed activatable groups such as hydroxyl or carboxyl groups. This activatable groups also allow easy covalent conjugation of a biomolecule of interest to a nanocrystal that has formed a host guest complex with the host molecule.

Every known nanocrystal can be employed in the present invention. In embodiments, in which no element A is present, the nanocrystal consists only of a metal such as gold, silver, copper (subgroup Ib), titanium (subgroup IVb), terbium (subgroup IIIb), cobalt, platinum, rhodium, ruthenium (subgroup VIIIb), lead (main group IV) or an alloy thereof. In this respect, it is noted that if in the following, the invention is illustrated with reference only to nanocrystals comprising an counter element A, it is clear that nanocrystals consisting of a pure metal or a metal alloy can used in all these embodiments as well. A nanocrystal used in the present invention may be a well known core-shell nanocrystal (quantum dot) such as a binary nanocrystal formed from metals such as Zn, Cd, Hg (subgroup IIb), Mg (main group II), Mn (main group VIIb), Ga, In, Al, (main group III) Fe, Co, Ni (subgroup VIIb), Cu, Ag, or Au (subgroup Ib). The nanocrystal may be any group II-VI semiconductor nanocrystal, wherein the core and/or the shell includes CdS, CdSe, CdTe, MgTe, ZnS, ZnSe, ZnTe, HgS, HgSe, or HgTe. The nanocrystal may also be any group III-V semiconductor nanocrystal wherein the core and/or the shell includes GaN, GaP, GaAs, GaSb, InN, InP, InAs, InSb, AlN, AlP, AlAs, AlSb. Specific examples of core shell nanocrystals that can be used in the present invention include, but are not limited to, (CdSe)-nanocrystals having a ZnS shell ((CdSe)—ZnS nanocrystals) or (CdS)—ZnS-nanocrystals.

However, the invention is by no means limited to the use of the above-described core shell nanocrystals. For example, in a further embodiment the nanocrystal that is to be rendered water soluble can be a nanocrystal consisting of a homogeneous ternary alloy having the composition M1₁₋ₓM2ₓA, wherein

a) M1 and M2 are independently selected from an element of subgroup IIb, subgroup VIIa, subgroup VIIIa, subgroup Ib or main group II of the periodic system of the elements (PSE), when A represents an element of the main group VI of the PSE, or
b) M1 and M2 are both selected from an element of the main group (III) of the PSE, when A represents an element of the main group (V) of the PSE.

In another embodiment a nanocrystal consisting of a homogeneous quaternary alloy can be used. Quaternary alloys of this type may have the composition M1₁₋ₓM2ₓA_{y}B_{1-y}, wherein

a) M1 and M2 are independently selected from an element of subgroup IIb, subgroup VIIa, subgroup VIIIa, subgroup Ib or main group II of the periodic system of the elements (PSE), when A and B both represent an element of the main group VI of the PSE, or
b) M1 and M2 are independently selected from an element of the main group (III) of the PSE, when A and B both represent an element of the main group (V) of the PSE.

Examples of this type of homogenous ternary or quaternary nanocrystals have been described in Zhong et al, J. Am. Chem. Soc, 2003 125, 8598-8594, Zhong et al, J. Am. Chem. Soc, 2003 125, 13559-13553, and the International application WO 2004/054923.

Such ternary nanocrystals are obtainable by a process comprising forming a binary nanocrystal M1A by
i) heating a reaction mixture containing the element M1 in a form suitable for the generation of a nanocrystal to a suitable temperature T1, adding at this temperature the element A in a form suitable for the generation of a nanocrystal, heating the reaction mixture for a sufficient period of time at a temperature suitable for forming said binary nanocrystal MIA and then allowing the reaction mixture to cool, and
ii) reheating the reaction mixture, without precipitating or isolating the formed binary nanocrystal M1A, to a suitable temperature T2, adding to the reaction mixture at this temperature a sufficient quantity of the element M2 in a form suitable for the generation of a nanocrystal, then heating the reaction mixture for a sufficient period of time at a temperature suitable for forming said ternary nanocrystal M1₁₋ₓM2ₓA and then allowing the reaction mixture to cool to room temperature, and isolating the ternary nanocrystal M1₁₋ₓM2ₓA.

In these ternary nanocrystals the index x has a value of 0.001<x<0.999, preferably of 0.01<x<0.99, 0.1<0.9 or more preferred of 0.5<x<0.95. In even more preferred embodiments, x can have a value between about 0.2 or about 0.3 to about 0.8 or about 0.9. In the quaternary nanocrystals employed here, y has a value of 0.001<y<0.999, preferably of 0.01<y<0.99, or more preferably of 0.1<x<0.95 or between about 0.2 and about 0.8.

In some embodiments of the II-VI ternary nanocrystals, the elements M1 and M2 comprised therein are preferably independently selected from the group consisting of Zn, Cd and Hg. The element A of the group VI of the PSE in these ternary alloys is preferably selected from the group consisting of S, Se and Te. Thus, all combinations of these elements M1, M2 and A are within the scope of the invention. In some presently preferred embodiments nanocrystals used have the composition ZnₓCd₁₋ₓSe, ZnₓCd₁₋ₓS, ZnₓCd₁₋ₓTe, HgₓCd₁₋ₓSe, HgₓCd₁₋ₓTe, HgₓCd₁₋ₓS, ZnₓHg₁₋ₓSe, ZnₓHg₁₋ₓTe, and ZnₓHg₁₋ₓS.

In this respect, it is noted that the designation M1 and M2 can be used interchangeably throughout the present application, for example in an alloy comprising Cd and Hg, either of which can be named M1 or M2. Likewise, the designation A and B for elements of group V or VI of the PSE are used interchangeably; thus in a quaternary alloy of the invention Se or Te can both be named as element A or B.

In some preferred embodiments, the ternary nanocrystals used herein have the composition ZnₓCd₁₋ₓSe. Such nanocrystals are preferred in which x has a value of 0.10<x<0.90 or 0.15<x<0.85, and more preferably a value of 0.2<x<0.8. In other preferred embodiments the nanocrystals have the composition ZnₓCd₁₋ₓS. Such nanocrystals are preferred in which x has a value of 0.10<x<0.95, and more preferably a value of 0.2<x<0.8.

In case of III-IV nanocrystals of the invention, the elements M1 and M2 are preferably independently selected from Ga and Indium. The element A is preferably selected from P, As and Sb.

In accordance with the above description, every nanocrystal (quantum dot) can be used in the present invention as long as its surface can be reacted with a capping reagent which has a (terminal) group that has affinity for (the surface of) the core nanocrystal. Accordingly, the capping reagent typically forms a covalent bond with the surface of the nanocrystal. In case of a core-shell nanocrystal, the covalent bond is usually formed between the capping reagent and the shell of the nanocrystal. In case a homogenous ternary or quaternary nanocrystal as described in WO 2004/054923 is used, the covalent bond is formed between the surface of the homogenous core and the capping reagent. The capping agent can be either of substantially hydrophilic or substantially hydrophobic nature, depending, for example, on the hydrophobicity (or hydrophily) of the inner cavity of the host molecule. In this respect it is noted that within the meaning of term “(substantially) hydrophobic molecule” is also a molecule that in addition to hydrophobic parts can also comprise hydrophilic parts as long as these hydrophilic parts do not interfere with the formation of the host guest complex by the hydrophobic parts of the molecule (i.e. capping agent) with a host molecule having a hydrophobic internal cavity. Likewise, the term “(substantially) hydrophilic molecule” include a molecule that in addition to hydrophilic parts can comprise hydrophobic parts as long as these hydrophobic parts do not interfere with the formation of the host guest complex by the hydrophilic parts of the molecule (i.e. capping reagent) with a host molecule having a hydrophilic internal cavity.

In one embodiment the capping reagent that is used for the “surface capping” has the formula (I)

H_{A}X—Y—Z,

wherein X is a terminal group selected from S, N, P, or O═P, A is an integer from 0 to 3, Y is a moiety having at least three main chain atoms, and Z is a hydrophobic ending group that can form a host-guest inclusion complex with a suitable host molecule.

Typically, the moiety Y of the capping reagent comprises 3 to 50 main chain atoms. The moiety Y can principally comprise any suitable moieties that confer a predominantly hydrophobic character to this reagent. Examples of suitable moieties which can be used in Y comprise alkyl moieties such as CH₂—groups, cycloalkyl moieties such as cyclohexyl groups, ether moieties such as—OCH₂CH₂— groups, or aromatic moieties such as a benzene ring or a naphthalene ring, to name a few of them. The moiety Y can be straight chained, branched and can also have substitutions to the main chain atoms. Z may be a —CH₃ group, a phenyl group (—C₆H₅), a —SH group, a hydroxyl group (OH), an acid group (for example, —SO₃H, PO₃H or a —COOH), a basic group (for example, NH_{2 or} NHR1 with R═CH₃ or —CH₂—CH₃), a halogen (—Cl, —Br, —I, —F) —OH, —CH═CH₂, a trimethylsilyl group (—Si(Me)₃), a ferrocene group, or an adamantine group to name a few examples.

In some embodiments, compounds such as CH₃(CH₂)ₙCH₂SH, CH₃O(CH₂CH₂O)ₙCH₂SH, HSCH₂CH₂CH₂(SH)(CH₂)ₙCH₃, CH₃(CH₂)ₙCH₂NH₂, CH₃O(CH₂CH₂O)ₙCH₂NH₂; P((CH₂)ₙCH₃)₃, O═P((CH₂)ₙCH₃)₃, wherein n is an integer 30≧n≧6 are used as capping agent. In other embodiments n is an integer 30≧n≧8.

In this regard it is noted examples of capping reagents that provide more hydrophobic or substantially hydrophobic properties include, but are not limited to, 1-mercapto-6-phenyl hexane acid (HS—(CH₂)₆-Ph), 1,16-dimercapto-hexadecane (HS—(CH₂)—₁₆—SH), 18-mercapto-octadecylamine (HS—(CH₂)₁₈—NH₂), trioctylphosphine, or 6-mercapto-hexane (HS—(CH₂)₅—CH₃).

Exemplary capping reagents that provide more hydrophobic or substantially hydrophilic properties include, but are not limited to, 6-mercapto-hexanoic acid (HS—(CH₂)₆—COOH), 16-mercapto-hexadeconic acid (HS—(CH₂)—₁₆—COOH), 18-mercapto-octadecylamine (HS—(CH₂)₁₈—NH₂), 6-mercapto-hexylamine (HS—(CH₂)₆—NH₂), or 8-hydroxy-octylthiol HO—(CH2)₈—SH.

Any host molecule can be used in the present invention, as long it is able to react with the capping agent and confers water solubility to the complex formed between the capped nanocrystal and the host molecule. Typically, the host molecule is a water soluble compound that contains solvent exposed polar groups such as hydroxyl groups, carboxylate groups, sulfonate groups, phosphate groups, amine groups, carboxamide groups or the like.

Examples of suitable host molecules include, but are not limited to carbohydrates, cyclic polyamines, cyclic peptides, crown ethers, dendrimers and the like.

Examples of cyclic polyamines that can be used as host molecules include tetraaza macrocyclic molecules such as 1,4,8,11-tetraazacyclotetradecane (also known as cyclam) and derivatives thereof such as 1,4,7,11-tetraazacyclotetradecane (isocyclam), 1-(2-aminomethyl)-1,4,8,11-tetraazacyclotetradecane (scorpiand), 1,4,8,11-tetraazacyclotetradecane-6,13-dicarboxylate which are described in Sroczynski and Grzejdaziak, J. Incl. Phenom. Macrocyclic Chem. 35, 251-260, 1999, or Bernhardt et al., J. Aus. Chem., 56, 679-684, 2003, hexaza macrocyclic complexes, (Hausmann, J. et al., Chemistry, A European Journal, 2004, 10, 1716; Piotrowski, T. et al., Electroanalysis, 2000, 12, 1397), or octaza macrocyclic compounds (Kobayashi, K. et al., J. Am. Chem. Soc. 1992, 114, 1105), for example. The octaza macrocyclic compounds described by Kobayashi, K. et al, supra are also one example of compounds that are suitable for accommodation of polar guest molecules (for example, hydrophilic capping agents). It is also possible to employ a cyclic polyamine which may only be water soluble to a limited extent, for example, 5,5,7,12,14,14-hexamethyl-1,4,8,11-tetraazacyclotetradecane (Me₆cylcam) and to modify it with substituents that provide polar groups such as carboxylate or sulfonate groups. Other examples of macrocyclic amines that can be used as host molecule are the compounds described in Odashima, K., Journal of Inclusion phenomena and molecular recognition in chemistry, 1998, 32, 165 (see for example, compounds 24 to 26 therein).

Examples of suitable calixarenes include 4-tert-Butylcalix[4]arenetetraacetic acid tetraethyl ester, tetragalactosylcalixarene as described in Dondoni et al, Chem. Eur., J. 3, 1774, 1997, tetragalactosylcalixarene (Davis, A. P. et al., Angew. Chem. Int. Edit., 1999, 38, 2979.) octaminoamide resorcin[4]-arenes (Kazakov, E. K. et al., Eur. J. Org. Chem., 2004, 3323.), 4-sulphonic calyx[n]-arenes (Yang, W. Z., J. Pharm. Pharmacology, 2004, 56, 703.), sulfonated thiacalix[4 or 6]-arene (Kunsasgi-Mate S., Tetrahedron Letters, 2004, 45, 1387), the calixarenes described in Kobayashi et al., J. Am. Chem. Soc. 116, 6081, 1994 and Yanagihara et al., J. Am. Chem. Soc. 114, 10307, 1992.

Examples of cyclic peptides that can be used as host molecule in the present invention include, but are not limited to, the dicyclodipeptide bearing calixarenes that are described in Guo, W et al., Tetrahedron Letters, 2002, 43, 5665; or Peng Li et al., Current Organic Chemistry, 2002, 6.

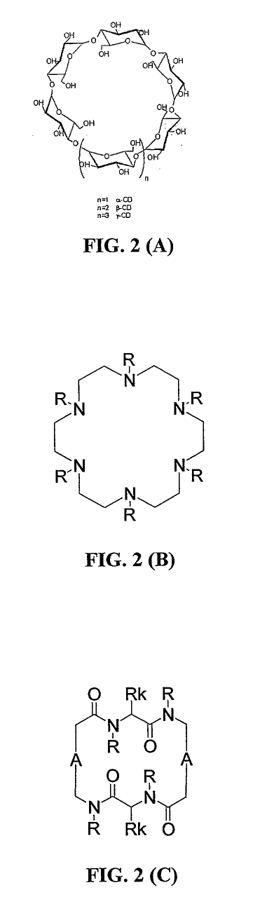

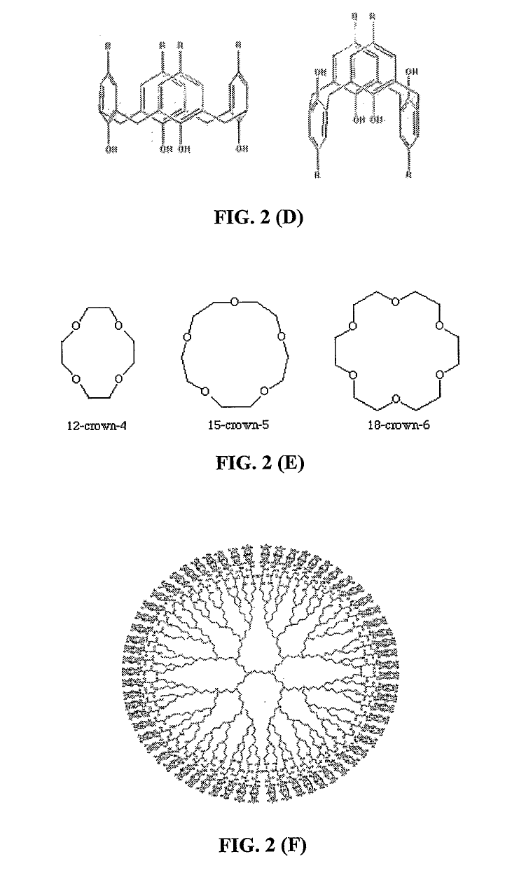

Crown ether that can employed as host molecule can have any ring size, for example, have a ring system comprising 8, 9, 10, 12, 14, 15, 16, 18 or 20 atoms of which some are typically heteroatoms such as O or S. Typical crown ethers used here include, but are not limited to, water soluble 8-Crown-4 compounds (wherein 4 indicates the number of heteroatoms), 9-Crown-3 compounds, 12-Crown-4 compounds, 15-Crown-5 compounds, 18-Crown-6 compounds, and 20-Crown-8 compounds (cf. also FIG. 2E). Examples of such suitable crown ethers include (18-Crown-6)-2,3,11,12 tetracarboxylic acid or 1,4,7,10-tetrazaacylcododecane-1,4,7,10 tetracarboxylic acid) to name only a few.

In principal, every water soluble dendrimer that provides a hydrophilic or hydrophobic cavity (depending on whether a hydrophobic or hydrophilic capping reagent is used) that is able to at least partially accommodate the capping reagent used in the present invention. Suitable classes of dendrimers include, but are not limited to, polypropylene imine dendrimers, polyamido amine dendrimers, poly aryl ether dendrimers, polylysine dendrimers, carbohydrate dendrimers and silicon dendrimers (reviewed in Boas and Heegard, Chem. Soc. Rev. 33, 43-63, 2004, for example).

In one embodiment, the nanocrystal of the present invention comprises a carbohydrate as host molecule. This carbohydrate host molecule may be, but is not limited to, an oligosaccharide, starch or a cyclodextrin molecule (cf. Davis and Wareham, Angew. Chem. Int. Edit. 38, 2979-2996, 1999).

In embodiments, in which the host molecule is an oligosaccharide, this oligosaccharide may comprise between 2, for example 6, and 20 monomer units in the main chain. These oligomers may be straight or branched chained. Examples of suitable oligosaccharides include, are not limited to 1,3-(dimethylene)benzenediyl-6,6′-O-(2,2′-oxydiethyl)-bis-(2,3,4-tri-O-acetyl-β-D-galactopyranoside), 1,3-(dimethylene)benzenediyl-6,6′-O-(2,2′-oxydiethyl)-bis-(2, 3,4-tri-O-methyl-β-D-galactopyranoside) Shizuma et at., J. Org. Chem. 2002, 67 4795), cyclotrikis-(1,2,3,4,5,6)-[α-D-glucopyranosyl)-(1,2,3,4)-α-D-glucopyranosyl], (Cescutti et al., Carbohydrate Research, 2000, 329, 647), acetylenosaccharides (Burli et al., Angew. Chem. Int. Edit. 1997, 36, 1852), or cyclic fructo-oligosaccharides (Takai et al., J. Chem. Soc. Chem. Commun., 1993, 53.).

If starch is used as host molecule the starch may have a molecular weight Mw of about 1,000 to about 6,000 Da. In some embodiments, the starch has a molecular weight Mw of about 4,000 Da≧Mw≧about 2,000 Da. Starches that can be used also include amylose, for example α-amylose or β-amylose.

Examples of cyclodextrins that are suitable as host molecule include α-cyclodextrin, β-cyclodextrin, γ-cyclodextrin, Dimethyl-α-cyclodextrin, Trimethyl-α-cyclodextrin, Dimethyl-β-cyclodextrin, Trimethyl-β-cyclodextrin, Dimethyl-γ-cyclodextrin, and Trimethyl-γ-cyclodextrin.

In accordance with the above disclosure, the present invention also refers in one embodiment to a method of preparing a water soluble nanocrystal comprising
reacting a nanocrystal having a core comprising at least one metal M1 selected from an element of subgroup IIB-VIB, IIIB-VB or IVB, main group II or main group III of the periodic system of the elements (PSE), and (in case binary nanocrystals are used) at least one element selected from an element of the main group V or VI of the periodic system of the elements, with a capping reagent, thereby attaching the capping agent to the surface of the core of the nanocrystal, and
then contacting the so obtained nanocrystal with a host molecule to form a host guest complex between the reagent and the water soluble host molecule. The (capping) reagent can either be of hydrophilic or hydrophobic nature. In case a pure metal nanocrystal or a homogenous ternary or quarternary nanocrystal as disclosed above is used, the same reaction can be carried out to prepare a nanocrystal of the invention.

This reaction is usually carried in two separate steps, with isolating the nanocrystals that carry the capping capping reagent on their surface. For example, nanocrystals that have been reacted with a reagent such as trioctylphosphine, trioctylphosphine oxide or mercaptoundecanoic acid can be isolated and stored for any desired time in a suitable organic solvent (for example, chloroform, methylene chloride, tetrahydrofuran, to name a few of them) before reacting them with the host molecule.

The host guest complex between the capped nanocrystal and the host molecule can be easily formed under various reaction conditions. For examples, complex formation may be formed by kneading a solution of the nanocrystals with an aqueous solution of the host molecule, for example a cyclodextrin solution, or by refluxing the nanocrystals with a respective aqueous solution. For the latter method, the nanocrystals present in an organic solvent may be transferred into aqueous solution after refluxing for an extended period of time (see Example 2, for instance). Other possibilities of complex formation include stirring or incubating nanocrystal suspensions in a solution of a host molecule such as a cyclodextrin solution or other host molecules at ambient temperature for a suitable period of time. A typical incubation time may range from about 1 to about 10 days, however, shorter or longer incubation times may of course also be used.

The invention is also directed to a further method of preparing a water soluble nanocrystal. This method comprises reacting a nanocrystal having a core comprising at least one metal M1 selected from an element of subgroup Ib, IIb, IIB-VIB, IIIB-VB or IVB, main group II or main group III of the periodic system of the elements (PSE), and at least one element A selected from an element of the main group V or VI of the periodic system of the elements, with a (capping) reagent. In this method the reagent is covalently linked to a water soluble host molecule that is selected from the group consisting of carbohydrates, cyclic polyamines, cyclic dipeptides, calixarenes, and dendrimers.

Also in this method, any capping reagent can be used that a terminal group that has affinity for the nanocrystal core. This means that the capping reagent may be a hydrophilic or a hydrophobic reagent. This either hydrophilic or hydrophobic capping reagent reacts with the nanocrystal via its terminal group and typically forms a covalent bond with the surface of the nanocrystal (cf. Masihul et al., J. Am. Chem. Soc. 2002, 43, 1132). In case of a core-shell nanocrystal, the covalent bond is usually formed with the shell of the nanocrystal and the capping reagent. In case a homogenous ternary or quaternary nanocrystal as described in WO 2004/054923 is used, the covalent bond will be formed between the surface of the homogenous core and the capping reagent.

In some embodiments of this method a capping reagent is employed that has the formula (II) H_{I}X—Y—B, wherein

X is a terminal group selected from S, N, P, or O═P,

I is an integer from 1 to 3,

Y is a moiety having at least three main chain atoms, and

B is the water soluble host molecule that is covalently linked to the capping reagent.

In this respect, it is noted that the covalent bond formed between the capping reagent and the host molecule can be any covalent bond, for example, a C—C bond, an ether bond (—O—), a thioether bond (—S—), an ester bond, an amide bond or an imide bond, to name only a few possibilities. The type of covalent bond usually depends only on the approach that is taken to link the host molecule with the capping reagent. For example, if the capping agent is an alkyl halide and the host molecule has free (or activated) hydroxyl or thiol groups, an ether or thioether bond is formed (see Examples 3 and 5, for instance). Alternatively, if the capping agent can provide an amine group for the covalent coupling and the host molecules has a reactive carboxyl group, an ester bond is formed. Accordingly, the choice of an appropriate combination of reactive groups for the covalent linkage of the host molecule and the capping reagent is within the knowledge of the person skilled in the art.

In this respect, it is also noted that is not necessary that the covalent bond is formed between the capping reagent and the host molecule (to yield a compound of formula (II) H_{I}X—Y—B) prior to the reaction with the nanocrystal. Rather, it this also within the scope of the present invention that the capping reagent is first reacted with the nanocrystal and then the covalent bond between the capping reagent and the host molecule is formed.

In one embodiment of this method, a capping reagent used has the formula:

H_{A}X—Y—Z

wherein X is a terminal group selected from S, N, P, or O═P, A is an integer from 0 to 3, Y is a moiety having at least three main chain atoms. Typically, the moiety Y of the (capping) reagent comprises 3 to 50 main chain atoms. The moiety Y can principally comprise any suitable moieties that confer a predominantly hydrophobic character to this reagent. Examples of suitable moieties which can be used in the moiety Y comprise alkyl moieties such as CH₂-groups, cycloalkyl moieties such as cyclohexyl groups, ether moieties such as —OCH₂CH₂— groups, or aromatic moieties such as a benzene ring or a naphthalene ring, to name a few of them. Y can be straight chained, branched and can also have substitutions to the main chain atoms. Z may be any functional group that can covalently couple to the host molecule, for example a —SH group, a hydroxyl group (OH), an acid group (for example, —SO₃H, PO₃H or a —COOH), a basic group (for example, NH_{2 or} NHR1 with R═CH₃ or —CH₂—CH₃), or a halogen (—Cl, —Br, —I, —F) to name only a few examples.

The present invention further refers to a nanocrystal, as disclosed here, conjugated to a molecule having binding affinity for a given analyte. By conjugation to a molecule having binding affinity for a given analyte, a marker compound or probe is formed. In this probe the nanocrystal of the invention serves as a label or tag which emits radiation, for example in the visible or near infrared range of the electromagnetic spectrum, that can be used for the detection of a given analyte.

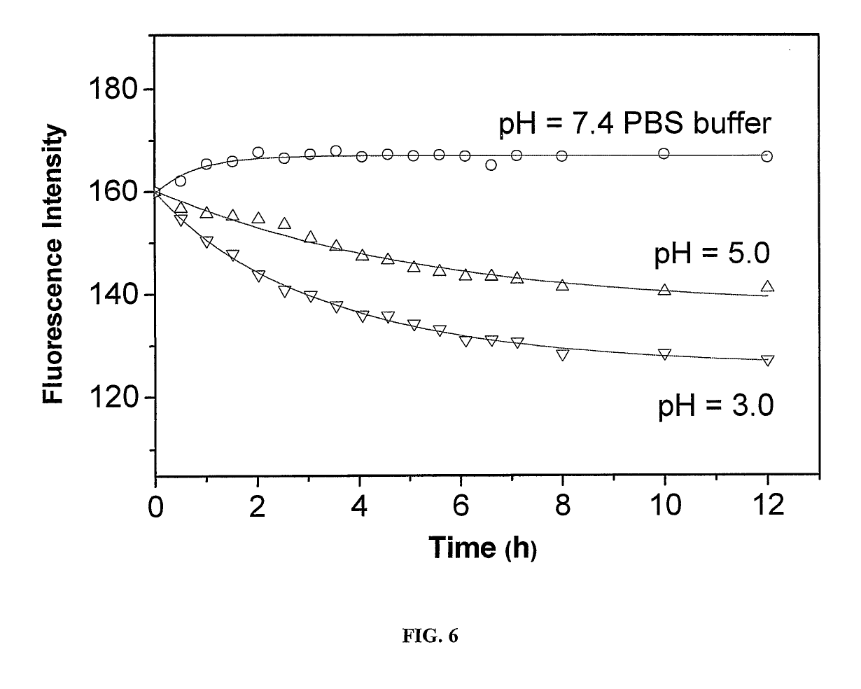

In principle every analyte can be detected for which a specific binding partner exists that is able to at least somewhat specifically bind to the analyte. The analyte can be a chemical compound such as a drug (e.g. Aspirin® or Ribavirin), or a biochemical molecule such as a protein (for example, an antibody specific for troponin or a cell surface protein) or a nucleic acid molecule. When coupled to an appropriate molecule with binding affinity (which is also referred to as the analyte binding partner) for an analyte of interest, such as Ribavirin, the resulting probe can be used for example in a fluorescent immunoassay for monitoring the level of the drug in the plasma of a patient. In case of troponin, which is a marker protein for damage of the heart muscle, and thus in general for a heart attack, a conjugate containing an anti-troponin antibody and an inventive nanocrystal can be used in the diagnosis of heart attack. In case of an conjugate of the inventive nanocrystals with an antibody that it specific for a tumor associated cell surface protein, this conjugate may be used for tumor diagnosis or imaging. Another example is a conjugate of the nanocrystal with streptavidin (cf. FIG. 6).

The analyte can also be a complex biological structure including but not limited to a virus particle, a chromosome or a whole cell. For example, if the analyte binding partner is a lipid that attaches to a cell membrane, a conjugate comprising a nanocrystal of the invention linked to such a lipid can be used for detection and visualization of a whole cell. For purposes such as cell staining or cell imaging, a nanocrystal emitting visible light is preferably used. In accordance with this disclosure the analyte that is to be detected by use of a marker compound that comprises a nanoparticle of the invention conjugated to an analyte binding partner is preferably a biomolecule.

Therefore, in a further preferred embodiment, the molecule having binding affinity for the analyte is a protein, a peptide, a compound having features of an immunogenic hapten, a nucleic acid, a carbohydrate or an organic molecule. The protein employed as analyte binding partner can be, for example, an antibody, an antibody fragment, a ligand, avidin, streptavidin or an enzyme. Examples of organic molecules are compounds such as biotin, digoxigenin, serotonine, folate derivatives and the like. A nucleic acid may be selected from, but not limited to, a DNA, RNA or PNA molecule, a short oligonucleotide with 10 to 50 bp as well as longer nucleic acids.

When used for the detection of biomolecules a nanocrystal of the invention can be conjugated to the molecule having binding activity via surface exposed groups of the host molecule. For this purpose, a surface exposed group such as an amine, hydroxyl or carboxylate group may be reacted with a linking agent. A linking agent as used herein, means any compound that is capable of linking a nanocrystal of the invention to a molecule having such binding affinity. Examples of the types of linking agents which may be used to conjugate a nanocrystal to the analyte binding partner are bi-functional linking reagents such as the bis-maleimide cross-linking reagents, the disulfide exchange cross-linking reagents, and the bis-N-hydroxysuccinimide ester cross-linking reagents. Examples of the suitable linking reagents are N,N′-1,4-phenylenedimaleimide, bismaleimidoethane, dithiobis-maleimdoethane, 1,11-bis-maleimidotetraethyleneglycol, C-6 bis disulfides, C-9 bis disulfides, disuccinimidyl glutarate, disuccinimidyl suberate, ethyleneglycol bis-(succinimidylsuccinate). However, if a nanocrystal of the invention is used, which comprises a capping reagent that is covalently linked to a water soluble host molecule, the host molecule can form a conjugate with a suitable linking agent (that may before or after the host guest complex formation) coupled to a selected molecule having the wished binding affinity. For example, if a cyclodextrin is used a host molecule suitable, linking agents include, but are not limited to, ferrocene derivatives, adamantan compounds, polyoxyethylene compounds, aromatic compounds all of which have a suitable reactive group for forming a covalent bond with the molecule of interest (cf. FIG. 6).

Furthermore, the invention is also directed to a composition containing at least one type of water-soluble nanocrystal as defined here. The nanocrystal may be incorporated into a plastic bead, a magnetic bead or a latex bead. Furthermore, a detection kit containing a nanocrystal as defined here is also part of the invention.

The invention is further illustrated by the following non-limiting examples and the attached drawings in which:

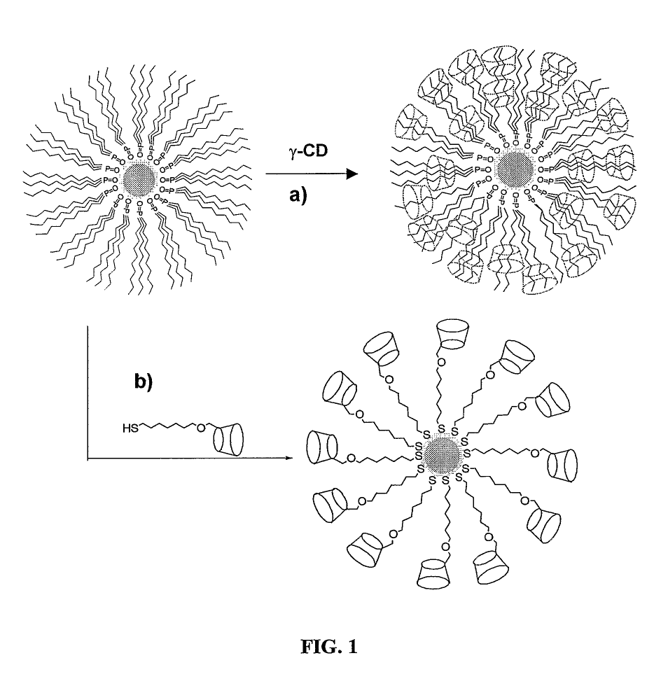

FIG. 1 is a schematic representation of water soluble nanocrystals of the invention which either have attached a hydrophobic reagent to the surface of the core of the nanocrystal which forms a host guest complex with cyclodextrin (CD) (scheme a)) or which have attached a hydrophobic agent that is covalently linked to a water soluble host molecule (scheme b));

FIG. 2 shows a schematic presentation of the structure of exemplary cyclodextrins (FIG. 2a), cyclic polyamines (FIG. 2b,), cyclic (di)peptides (FIG. 2c), calixarenes (FIG. 2d), crown ethers (FIG. 2e), and dendrimers (FIG. 20 that can be used as host molecules in the present invention;

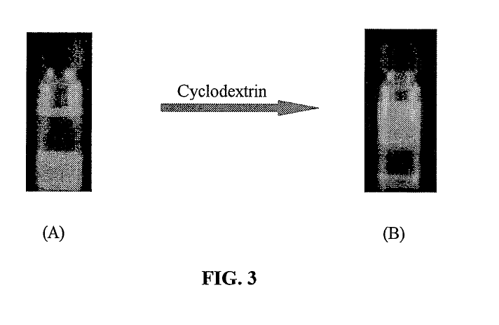

FIG. 3 shows the phase transfer of TOP-capped CdSe/ZnS core shell nanocrystals from chloroform (FIG. 3a) to aqueous solution (FIG. 3b) that is caused by the addition of γ-cyclodextrin;

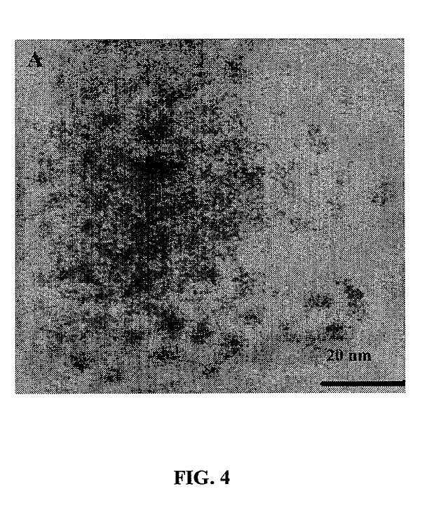

FIG. 4 shows a TEM micrograph of CdSe/ZnS core shell nanocrystals forming a host guest complex with γ-cyclodextrin;

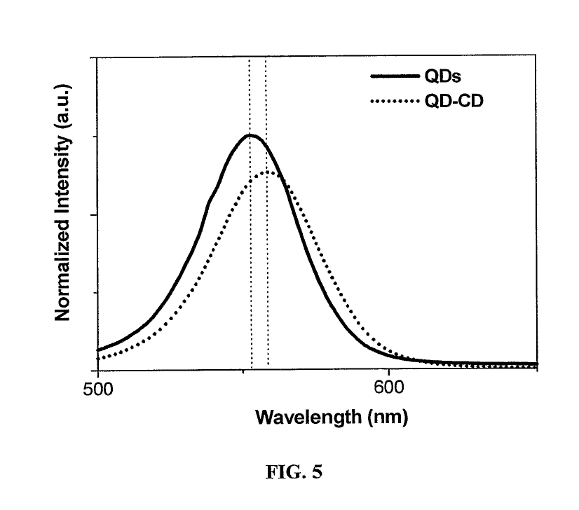

FIG. 5 shows the fluorescence intensity of CdSe/ZnS core shell nanocrystals of the invention compared to the starting nanocrystals before formation of the host guest complex;

FIG. 6 shows the effect of the pH on the photoluminescence of CdSe/ZnS core shell nanocrystals of the invention forming a host guest complex with γ-cyclodextrin;

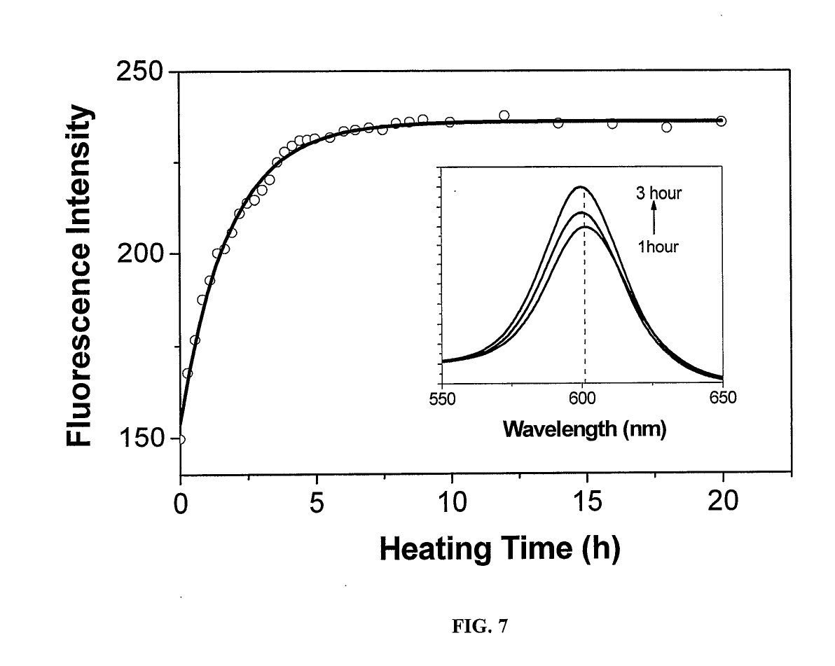

FIG. 7 shows the thermal stability of CdSe/ZnS core shell nanocrystals of the invention at 50° C.;

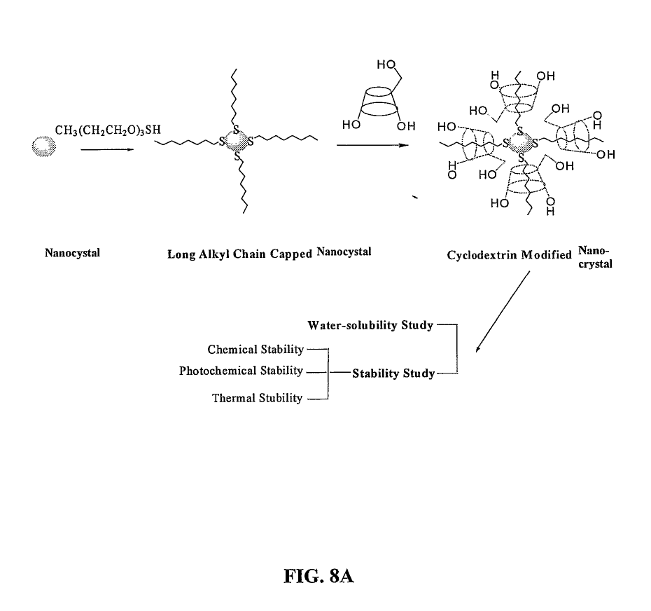

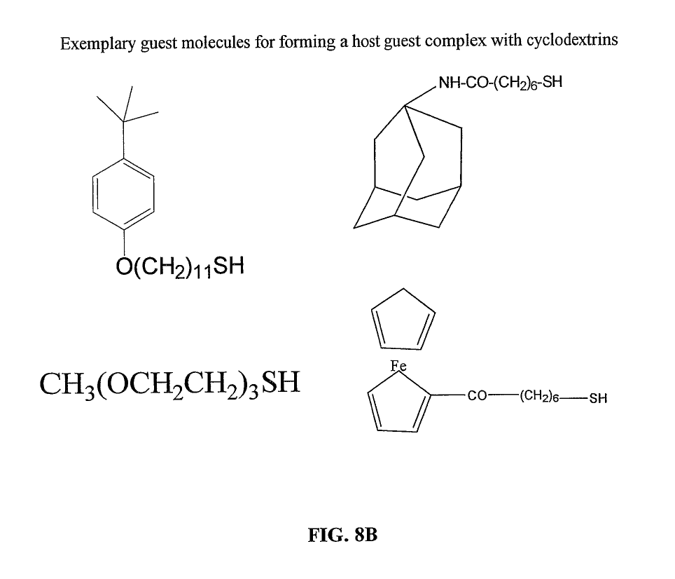

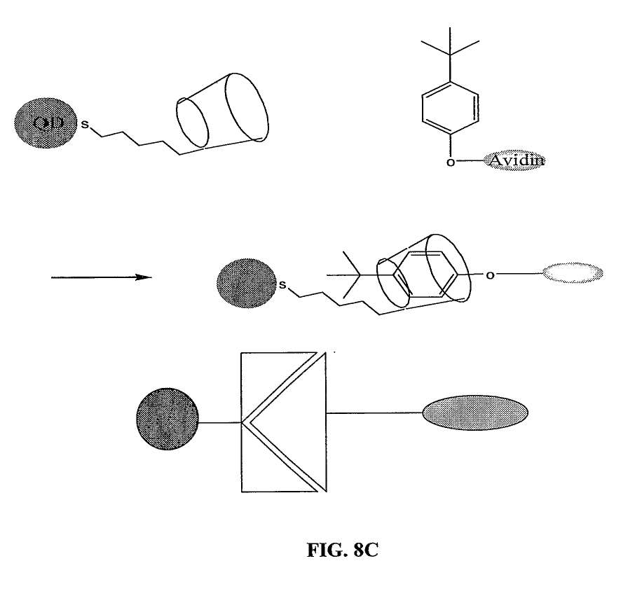

FIG. 8 shows a schematic drawing of the preparation of a nanocrystal of the invention comprising a host guest-complex, wherein the host molecule has free reactive groups that can used of preparation of a conjugate (FIG. 8a). FIG. 8 also shows examples of ligands that can form a host guest complex with a host molecule such as cyclodextrin for preparation of conjugates of the water soluble nanocrystals of the invention (FIG. 8b) as well as a schematic drawing of a conjugate of a nanocrystal of the invention with streptavidin (FIG. 8c).

### EXAMPLE 1

### Preparation of TOPO Capped (CdSe)—ZnS Nanocrystals (Quantum Dots, QD)

Trioctylphosphine (TOP)/Trioctylphosphine oxide (TOPO) capped CdSe nanocrystals were prepared as follows. TOPO (30 g) was placed in a flask and dried under vacuum (˜1 Torr) at 180° C. for 1 hour. The flask was then filled with nitrogen and heated to 350° C. In an inert atmosphere drybox the following injection solution was prepared: CdMe₂ (200 ml), 1 M TOPSe solution (4.0 ml), and TOP (16 ml). The injection solution was thoroughly mixed, loaded into a syringe, and removed from the drybox.

The heat was removed from the reaction and the reaction mixture was transferred into vigorously stirring TOPO with a single continuous injection. Heating was resorted to the reaction flask and the temperature was gradually raised to 260-280° C. After the reaction, the reaction flask was allowed to cool to ˜60° C., and 20 ml of butanol were added to prevent solidification of the TOPO. Addition of large excess of methanol causes the particles to flocculate. The flocculate was separated from the supernatant liquid by centrifugation; the resulting powder can be dispersed in a variety of organic solvents to produce an optically clear solution.

A flask containing 5 g of TOPO was heated to 190° C. under vacuum for several hours then cooled to 60° C. after which 0.5 ml trioctylphosphine (TOP) was added. Roughly 0.1-0.4 μmols of CdSe dots dispersed in hexane were transferred into the reaction vessel via syringe and the solvent was pumped off. Diethyl zinc (ZnEt₂) and hexamethyldisilathiane ((TMS)₂S) were used as the Zn and S precursors, respectively. Equimolar amount of the precursors were dissolved in 2-4 ml TOP inside an inert atmosphere glove box. The precursor solution was loaded into a syringe and transferred to an additional funnel attached to the reaction flask. After the addition was completed, the mixture was cooled to 90° C. and left stirring for several hours. Butanol was added to the mixture to prevent the TOPO from solidifying upon cooling to room temperature.

### EXAMPLE 2

### Preparation of Water-Soluble Nanocrystals by Formation of Host-Guest Complex with γ-cyclodextrin

The nanocrystals obtained in Example 1 having a hydrophobic capping with TOP/TOPO were dissolved into 200 μl of a mixture of chloroform/hexane (1:1). About 0.5 g of γ-cyclodextrin and the nanocrystal solution were added to a solution of 20 ml deionized water. The mixture was refluxed for about 8 hour until a cloudy solution formed. A rotary evaporator was used to remove most of the water, and then the formed host-guest inclusion complex was isolated by centrifugation. The collected solid was further washed with water to remove the free cyclodextrins molecules. The so obtained nanocrystals that had formed a host guest complex with cyclodextrins via TOP/TOPO were stored in solid state. They can easily be transferred into water by dissolving them in water by means of ultrasonic treatment. The nanocrystals which are protected by the host/guest complex were found to be stable in the solid state for a relatively long time.

The formation of the water soluble γ-CD modified quantum dots by formation of a host guest complex could be followed optically. When adding γ-cyclodextrin to a chloroform solution containing TOP/TOPO-capped CdSe/ZnS core shell nanocrystals, the formed nanocrystals migrated from the organic chloroform phase (FIG. 3a) to the aqueous solution (FIG. 3b).

The formation of γ-CD modified quantum dots was also confirmed by ¹H-NMR, FT-IR spectroscopy and XRD measurement (data not shown). Transmission electron microscopy (TEM) (FIG. 4) and fluorescence images (cf. FIG. 5 for example) show that the quantum dots having formed host guest complexes with γ-cyclodextrin form high mono-dispersed particles. FIG. 5 in addition shows that the CdSe/ZnS core shell nanocrystals of the invention possess a higher fluorescence intensity after formation of the host complex (measured in water) than the unmodified TOP/TOPO-capped core shell nanocrystals (measured in CHCl₃), whereas the wavelengths of the emission maximum remained unchanged. The photoluminescence measurements of FIG. 6 shows that that the CdSe/ZnS core shell nanocrystals that have formed a host guest complex with γ-cyclodextrin are very stable in PBS puffer of pH 7.4 (open circles) (i.e. under physiological conditions), and even show a satisfying stability in aqueous solution of pH 5.0 (upwards pointing triangles) and pH 3.0, respectively (downwards pointing triangles). Finally, FIG. 7 illustrates that the CdSe/ZnS core shell nanocrystals after having formed a host guest complex with γ-cyclodextrin show a good thermal stability in aqueous solution when heated to 50° C.

### EXAMPLE 3

### Preparation of (β-cyclodextrin monoalkylthiol (octanthiol)

LiH (5 mmol) was added to a solution of dried tert-Butyldimethylsilyl (TBDMS) protected cyclodextrin (TBDMSCD) (2.2 mmol) in dry THF (50 ml) and refluxed for about 3 hours. Then triphenyl methanol protected 8-bromo-1-octanthiol (4 mmol) was added and refluxed overnight. The solvent was removed in vacuo and the residue was dissolved in chloroform. The solution was washed with diluted HCl solution, then brine, and dried. Purification was carried out by column chromatography on silica (mesh 200-400). The obtained solid was dissolved into TFA (10 ml). When the solution became colorless, remaining trace of the acid under reduced pressure and the crude reaction product was dissolved in water. For purification, the cyclodextrin octanthiol was washed with diethyl ether to remove the unreacted starting materials. After lyophilization, the product was obtained as a powder in a yield of 21%. ¹HNMR (D₂O, δ, ppm): 5.1, 3.9-3.2, 2.4, 1.5-1.0.

### EXAMPLE 4

### Preparation of Water-Soluble Quantum Dots by Ligand Exchange with Cyclodextrin Monoalkylthiol

Purification of the TOP/TOPO capped quantum dots was done by dissolving of the quantum dots in chloroform and precipitated from acetone and methanol as described in Zhong et al., J. Am. Chem. Soc. 125, 8589, 2003. The obtained quantum dots were dissolved in dry chloroform to form a clear solution. Under stirring, an excess of cyclodextrin monoalkylthiol prepared in Example 3 was added in portion. Each time, cyclodextrin monoalkylthiol (octanthiol) was added till the solution became clear. After the addition was completed, the reaction mixture was kept stirring at room temperature overnight. The solvent was removed in vacuo and the obtained solid was washed with diethyl ether to remove the free cyclodextrin monoalkylthiol. The resulting powder was collected and further purified by centrifugation from a pure water solution. After lyophilization, the product was collected and characterized by ¹HNMR. ¹HNMR (D₂O, δ, ppm): 5.1, 4.1-3.2, 2.3, 1.5-1.0, 0.9-0.8.

### EXAMPLE 5

### Preparation of 6-thio-β-cyclodextrin

Per-6-iodo-β-cyclodextrin (1 g) was dissolved in DMF (10 ml); thiourea (0.301 g) was then added and the reaction mixture heated to 70° C. under a nitrogen atmosphere. After 19 h, the DMF was removed under reduced pressure to give a yellow oil, which was dissolved in water (50 ml). Sodium hydroxide (0.26 g) was added and the reaction mixture heated to gentle reflux under a nitrogen atmosphere. After 1 h, the resulting suspension was acidified with aqueous KHSO₄ and the resulting precipitate filtered off, washed thoroughly with distilled water, and then dried. To remove the last traces of DMF, the product was suspended in water (50 mL) and the minimum amount of potassium hydroxide added to give a clear solution; the product was then reprecipitated by acidifying with aqueous KHSO₄. The resulting fine precipitate was carefully filtered off and dried under vacuum over P₂O₅ to yield per-6-thio-cyclodextrin (65%) as an off-white powder. ¹H NMR (DMSO, δ, ppm) 2.16, 2.79, 3.21, 3.36-3.40, 3.60, 3.68, 4.95, 5.83, 5.97.

### EXAMPLE 6

### Preparation of Water-Soluble Quantum Dots Capped by 6-thio-β-cyclodextrin

Purification of the TOP/TOPO capped quantum dots is similar to the procedure described in Examples 2 and 4. The obtained quantum dots were dissolved in dry pyridine to form a clear solution. Under stirring, 6-thio-β-cyclodextrin was added. 10 minutes later, the reaction became clear. Stirring was continued at room temperature overnight. Most of the solvent was removed and then 50 ml diethyl ether was added. A white precipitate was collected and rinsed again with diethyl ether. The obtained powder was filtered off and dried. ¹HNMR (DMSO, δ, ppm): 5.8, 5.1, 4.1-3.2, 2.6, 2.2, 1.5-1.0.

### EXAMPLE 7

### Preparation of Conjugates Comprising a Nanocrystal of the Invention

FIG. 8a shows a reaction scheme for preparing a nanocrystal of the invention that comprises a host-guest complex of a capping reagent with a suitable host molecule.

As explained above, a suitable capping reagent that is bonded to the outer surface of the nanocrystal may be a thiol compound with a long alkyl chain or a polyoxyalkyl chain. Such a capped nanocrystal may be reacted with a host molecule such as a cyclodextrin leading to a highly stably a water soluble nanocrystal. For preparation of a conjugate of the nanocrystal that may be used as a diagnostic tool such a host molecule can either be conjugated with a ligand of interest such as biotin, digoxigenin, a small molecule drug or an protein such as streptavidin, avidin or an antibody, to name only a few examples.

The conjugate can be prepared by reacting a free reactive group such as a solvent exposed hydrophilic group (e.g. an —OH, COOH or NH₂ group) with the ligand of interest (cf. FIG. 8a). The conjugate may also be prepared by forming a further host guest complex between the guest molecule and a suitable host molecule that is linked to the ligand of interest. Exemplary host molecules that can be used for forming such a (second) host guest complex with a cyclodextrin compound, for example, are shown in FIG. 8b. It is noted in this regard that it is within the knowledge of the average person skilled in the art to select the appropriate guest for the chosen host molecule. This approach of forming a conjugate of a nanocrystal of the invention via a host guest complex is illustrated by the streptavidin conjugate shown in FIG. 8c.

1. A water soluble nanocrystal having a core comprising
at least one metal M1 selected from an element of subgroup Ib, subgroup IIb, subgroup IIIb, subgroup IVb, subgroup Vb, subgroup VIb, subgroup VIIb, subgroup VIIIb, main group II, main group III or main group IV of the periodic system of the elements (PSE),
wherein a capping reagent is attached to the surface of the core of the nanocrystal, and
wherein the capping reagent forms a host guest complex with a water soluble host molecule.

2. A water soluble nanocrystal having a core comprising
at least one metal M1 selected from an element of subgroup Ib, subgroup IIb, subgroup IIIb, subgroup IVb, subgroup Vb, subgroup VIb, subgroup VIIb, subgroup VIIIb, main group II, main group III or main group IV of the periodic system of the elements (PSE), and
at least one element A selected from an element of the main group V or VI of the periodic system of the elements,
wherein a capping reagent is attached to the surface of the core of the nanocrystal, and
wherein the capping reagent forms a host guest complex with a water soluble host molecule.

3. The nanocrystal of claim 2, wherein the capping reagent is a hydrophobic or a hydrophilic agent.

4. The nanocrystal of claim 3, wherein the capping reagent has a terminal group that has affinity for the nanocrystal core.

5. The nanocrystal of claim 3, wherein the capping reagent has the formula (I)
HₐX—Y—Z,
wherein
X is a terminal group selected from S, N, P, or O═P,
A is an integer from 0 to 3,
Y is a moiety having at least three main chain atoms, and
Z is a hydrophobic ending group.

6. The nanocrystal of claim 5, wherein the moiety Y of the capping reagent comprises 3 to 50 main chain atoms.

7. The nanocrystal of claim 6, wherein Y comprises alkyl moieties, cycloalkyl moieties, ether moieties or aromatic moieties.

8. The nanocrystal of claim 5, wherein the capping agent is selected from the group consisting of CH₃(CH₂)ₙCH₂SH, CH₃O(CH₂CH₂O)ₙCH₂SH, HSCH₂CH₂CH₂(SH)(CH₂)ₙCH₃, CH₃(CH₂)ₙCH₂NH₂, CH₃O(CH₂CH₂O)ₙCH₂NH₂; P((CH₂)ₙCH₃)₃, O═P((CH₂)ₙCH₃)₃, wherein n is an integer ≧6.

9. The nanocrystal of claim 8, wherein n is an integer ≧8.

10. The nanocrystal of claim 2, wherein the water soluble host molecule is a compound containing solvent exposed polar groups.

11. The nanocrystal of claim 10, wherein the host molecule is selected from the group consisting of carbohydrates, cyclic polyamines, cyclic peptides, calixarenes, crown ethers, and dendrimers.

12. The nanocrystal of claim 11, wherein the carbohydrate is selected from the group consisting of an oligosaccharide, starch and a cyclodextrin.

13. The nanocrystal of claim 12, wherein the starch is α-amylose or β-amylose.

14. The nanocrystal of claim 12, wherein the cyclodextrin is selected from the group consisting of α-cyclodextrin, β-cyclodextrin, γ-cyclodextrin, Dimethyl-α-cyclodextrin, Trimethyl-α-cyclodextrin, Dimethyl-β-cyclodextrin, Trimethyl-β-cyclodextrin, Dimethyl-γ-cyclodextrin, and Trimethyl-γ-cyclodextrin.

15. The nanocrystal of claim 12, wherein the oligosaccharide comprises 2 to 20 monomer units.

16. The nanocrystal of claim 2, wherein the nanocrystal is a core-shell nanocrystal.

17. The nanocrystal of claim 16, wherein the metal is selected from the group consisting of Zn, Cd, Hg, Mn, Fe, Co, Ni, Cu, Ag, and Au.

18. The nanocrystal of claim 16, wherein the element A is selected from the group consisting of S, Se, and Te.

19. The nanocrystal of Claim 16, wherein the nanocrystal is a core shell nanocrystal selected from the group consisting of CdS, CdSe, MgTe, CdTe, ZnS, ZnSe, ZnTe, HgS, HgSe, and HgTe.

20. The nanocrystal of claim 2, wherein the nanocrystal is consisting of a homogeneous ternary alloy having the composition M1₁₋ₓM2ₓA, wherein
a) M1 and M2 are independently selected from an element of subgroup IIb, subgroup VIIa, subgroup VIIIa, subgroup Ib or main group II of the periodic system of the elements (PSE), when A represents an element of the main group VI of the PSE, or
b) M1 and M2 are both selected from an element of the main group (III) of the PSE, when
A represents an element of the main group (V) of the PSE, obtainable by a process comprising
i) forming a binary nanocrystal MIA by heating a reaction mixture containing the element M1 in a form suitable for the generation of a nanocrystal to a suitable temperature T1, adding at this temperature the element A in a form suitable for the generation of a nanocrystal, heating the reaction mixture for a sufficient period of time at a temperature suitable for forming said binary nanocrystal M1A and then allowing the reaction mixture to cool, and
ii) reheating the reaction mixture, without precipitating or isolating the formed binary nanocrystal M1A, to a suitable temperature T2, adding to the reaction mixture at this temperature a sufficient quantity of the element M2 in a form suitable for the generation of a nanocrystal, then heating the reaction mixture for a sufficient period of time at a temperature suitable for forming said ternary nanocyrstal M1₁₋ₓM2ₓA and then allowing the reaction mixture to cool to room temperature, and isolating the ternary nanocrystal M1₁₋ₓM2ₓA.

21. The nanocrystal of claim 20 with 0.001<x<0.999.

22. The nanocrystal of claim 20 with 0.01<x<0.99.

23. The nanocrystal of claim 20 with 0.5<x<0.95.

24. The nanocrystal of claim 20, wherein the elements M1 and M2 are independently selected from the group consisting of Zn, Cd, Hg, Mn, Fe, Co, Ni, Cu, Ag, and Au.

25. The nanocrystal of claim 20, wherein the element A is selected from the group consisting of S, Se and Te.

26. The nanocrystal of claim 20 having the composition ZnₓCd₁₋ₓSe or ZnₓCd₁₋ₓS.

27. A method of preparing a water soluble nanocrystal comprising reacting a nanocrystal having a core comprising at least one metal M1 selected from an element selected from subgroup Ib, subgroup IIb, subgroup IIIb, subgroup IVb, subgroup Vb, subgroup VIb, subgroup VIIb, subgroup VIIIb, main group II, main group III or main group IV with a capping reagent, thereby attaching the capping reagent to the surface of the core of the nanocrystal, and
then contacting the so obtained nanocrystal with a host molecule to form a host guest complex between the capping reagent and the water soluble host molecule.

28. A method of preparing a water soluble nanocrystal comprising reacting a nanocrystal having a core comprising at least one metal M1 selected from an element of subgroup Ib, subgroup IIb, subgroup IIIb, subgroup IVb, subgroup Vb, subgroup VIb, subgroup VIIb, subgroup VIIIb, main group II, main group III or main group IV of the periodic system of the elements (PSE), and
at least one element A selected from an element of the main group V or VI of the periodic system of the elements,
with a capping reagent, thereby attaching the capping reagent to the surface of the core of the nanocrystal, and then
contacting the so obtained nanocrystal with a host molecule to form a host guest complex between the reagent and the water soluble host molecule.

29. The method of claim 28, wherein the capping reagent is a hydrophobic or a hydrophilic agent.

30. The method of claim 29, wherein the capping reagent has a terminal group that has affinity for the nanocrystal core.

31. The method of any of claim 28, wherein a capping reagent is used that has the formula (I)
HₐX—Y—Z,
wherein
X is a terminal group selected from S, N, P, or O═P,
A is an integer from 0 to 3,
Y is a moiety having at least three main chain atoms, and
Z is a hydrophobic ending group.

32. The method of claim 31, wherein the moiety Y of the capping agent comprises 3 to 50 main chain atoms.

33. The method of claim 32, wherein Y comprises alkyl moieties, cycloalkyl moieties, ether moieties, or aromatic moieties.

34. The method of claim 31, wherein the reagent used is selected from the group consisting of CH₃(CH₂)ₙCH₂SH, CH₃O(CH₂CH₂O)ₙCH₂SH, HSCH₂CH₂CH₂(SH)(CH₂)ₙCH₃; CH₃(CH₂)ₙCH₂NH₂, CH₃O(CH₂CH₂O)ₙCH₂NH₂; P((CH₂)ₙCH₃)₃, and O═P((CH₂)ₙCH₃, wherein n is an integer ≧6.

35. The method of claim 29, wherein the water soluble host molecule used is a compound containing solvent exposed polar groups.

36. The method of claim 35, wherein the host molecule used is selected from the group consisting of carbohydrates, cyclic polyamines, cyclic peptides, calixarenes, crown ethers, and dendrimers.

37-40. (canceled)

41. The method of claim 35, wherein the host guest complex is formed by kneading, by refluxing, by stirring or incubating at ambient temperature for about 1 to about 10 days the nanocrystals with an aqueous solution of the host molecule.

42. A water soluble nanocrystal having a core comprising
at least one metal M1 selected from an element of subgroup Ib, subgroup IIb, subgroup IIIb, subgroup IVb, subgroup Vb, subgroup VIb, subgroup VIIb, subgroup VIIIb, main group II or main group III of the periodic system of the elements (PSE), and
at least one element A selected from an element of the main group V or VI of the periodic system of the elements, and,
wherein a capping reagent is attached to the surface of the core of the nanocrystal, and
wherein the capping reagent is covalently linked to a water soluble host molecule, and wherein the host molecule is selected from the group consisting of carbohydrates, cyclic polyamines, cyclic peptides, calixarenes, and dendrimers.

43. The nanocrystal of claim 42, wherein the capping reagent is a hydrophobic or a hydrophilic reagent having a terminal group that has affinity for the nanocrystal.

44. The nanocrystal of claim 42, wherein the capping reagent has the formula (II)
H₁X—Y—B (II),
wherein
X is a terminal group selected from S, N, P, or O═P,
1 is an integer from 1 to 3,
Y is a moiety having at least three main chain atoms, and
B is a water soluble host molecule.

45. The nanocrystal of claim 44, wherein the moiety Y of the capping agent comprises 3 to 50 main chain atoms.

46. The nanocrystal of claim 45, wherein Y comprises alkyl moieties, cycloalkyl moieties, ether moieties, or aromatic moieties.

47. The nanocrystal of claim 42, wherein the capping agent is selected from the group consisting of CH₃(CH₂)ₙCH₂SH, CH₃O(CH₂CH₂O)ₙCH₂SH, HSCH₂CH₂CH₂(SH)(CH₂)ₙCH₃, CH₃(CH₂)ₙCH₂NH₂, CH₃O(CH₂CH₂O)ₙCH₂NH₂; P((CH₂)ₙCH₃)₃, and O═P((CH₂)ₙCH₃)₃, wherein n is an integer ≧6.

48. The nanocrystal of claim 47, wherein n is an integer ≧8.

49. The nanocrystal of claim 42, wherein the carbohydrate used is selected from the group consisting of an oligosaccharide, starch and a cyclodextrin.

50. The nanocrystal of claim 49, wherein the starch is α-amylose or β-amylose.

51. The nanocrystal of claim 49, wherein the cyclodextrin used is selected from the group consisting of α-cyclodextrin, β-cyclodextrin, γ-cyclodextrin, Dimethyl-α-cyclodextrin, Trimethyl-α-cyclodextrin, Dimethyl-β-cyclodextrin, Trimethyl-β-cyclodextrin, Dimethyl-γ-cyclodextrin, and Trimethyl-γ-cyclodextrin.

52. The nanocrystal of claim 51, wherein the oligosaccharide comprises 6 to 20 monomer units.

53. A method of preparing a water soluble nanocrystal comprising
reacting a nanocrystal having a core comprising at least one metal M1 selected from an element of subgroup Ib, IIb, IIB-VIB, IIB-VB or IVB, main group II or main group III of the periodic system of the elements (PSE), and at least one element A selected from an element of the main group V or VI of the periodic system of the elements,
with a capping reagent, wherein the capping agent is covalently linked to a water soluble host molecule that is selected from the group consisting of carbohydrates, cyclic polyamines, cyclic dipeptides, calixarenes, and dendrimers.

54. The method of claim 53, wherein the reagent is a hydrophobic capping reagent or a hydrophilic capping reagent having a terminal group that has affinity for the nanocrystal core.

55. The method of claim 53, wherein the reagent has the formula (II)
H₁X—Y—B (II),
wherein
X is a terminal group selected from S, N, P, or O═P,
1 is an integer from 1 to 3,
Y is a moiety having at least three main chain atoms, and
B is the water soluble host molecule covalently linked to the reagent.

56. The method of claim 55, wherein the moiety Y of the capping agent comprises 3 to 50 main chain atoms.

57. The method of claim 56, wherein Y comprises alkyl moieties, cycloalkyl moieties, ether moieties, or aromatic moieties.

58. The method of claim 53, wherein the capping agent is selected from the group consisting of CH₃(CH₂)ₙCH₂SH, CH₃O(CH₂CH₂O)ₙCH₂SH, HSCH₂CH₂CH₂(SH)(CH₂)ₙCH₃, CH₃(CH₂)ₙCH₂NH₂, CH₃—O—(CH₂CH₂O)ₙCH₂NH₂; P((CH₂)ₙCH₃)₃, and O═P((CH₂)ₙCH₃)₃, wherein n is an integer ≧3.

59-60. (canceled)

61. A water soluble nanocrystal having a core comprising
at least one metal M1 selected from an element of subgroup Ib, IIb, IIB-VIB, IIIB-VB or IVB, main group II or main group III of the periodic system of the elements (PSE), and at least one element A selected from an element of the main group V or VI of the periodic system of the elements, and,
wherein a hydrophobic capping reagent is attached to the surface of the core of the nanocrystal, and
wherein the hydrophobic capping agent is covalently linked to a crown ether and wherein the hydrophobic reagent has the formula (I)
HₐX—Y—Z,
wherein
X is a terminal group selected from S, N, P, or O═P,
A is an integer from 0 to 3,
Y is a moiety having at least three main chain atoms, and
Z is a hydrophobic ending group.

62. The nanocrystal of claim 61, wherein the moiety Y of the capping reagent comprises 3 to 50 main chain atoms.

63. The nanocrystal of claim 62, wherein Y comprises alkyl moieties, cycloalkyl moieties, ether moieties, or aromatic moieties.

64. The nanocrystal of claim 62, wherein the hydrophobic reagent is selected from the group consisting of CH₃(CH₂)ₙCH₂SH, CH₃—O—(CH₂CH₂O)ₙCH₂SH, HSCH₂CH₂CH₂(SH)(CH₂)ₙCH₃; CH₃(CH₂)ₙCH₂NH₂, CH₃O(CH₂CH₂O)ₙCH₂NH₂; P((CH₂)ₙCH₃)₃, and O═P((CH₂)ₙCH₃)₃, wherein n is an integer ≧6.

65. The nanocrystal of claim 61, wherein the crown ether is a compound selected from the group consisting of 8-Crown-4 compounds, 9-Crown-3 compounds, 12-Crown-4 compounds, 15-Crown-5 compounds, 18-Crown-6 compounds, and 20-Crown-8 compounds.

66. A nanocrystal as defined in claim 2, conjugated to a molecule having binding affinity for a given analyte.

67. The nanocrystal of claim 66, wherein the molecule having binding affinity for a given analyte has binding affinity to a biomolecule.

68. The nanocrystal of claim 67, wherein the molecule having binding affinity for an analyte is a protein, a peptide, a compound having features of an immunogenic hapten, a nucleic acid, a carbohydrate or an organic molecule.

69. The nanocrystal of claim 67, wherein the nanocrystal is conjugated to said molecule having binding activity for an analyte via a covalent linking agent.

70. The nanocrystal of claim 67, wherein the nanocrystal is conjugated to said molecule having binding activity for an analyte via a ligand that is bound by the host molecule.

71. (canceled)

72. A nanocrystal as defined in claim 42, conjugated to a molecule having binding affinity for a given analyte.

73. A method of detecting an analyte, the method comprising
providing a probe formed by a nanocrystal according to claim 2 in that the nanocrystal is conjugated to a molecule having binding affinity for a given analyte, wherein the nanocrystal serves as a label,
contacting the probe with a sample suspected to comprise the analyte, and
detecting radiation emitted by the nanocrystal.

74. A method of detecting an analyte, the method comprising
providing a probe formed by a nanocrystal according to claim 42 in that the nanocrystal is conjugated to a molecule having binding affinity for a given analyte, wherein the nanocrystal serves as a label,
contacting the probe with a sample suspected to comprise the analyte, and detecting radiation emitted by the nanocrystal.
